(19) United States
(12) Patent Application Publication
(10) Pub. No.: US 2023/0128867 A1
(43) Pub. Date: Apr. 27, 2023

(54) MEMBRANE-ELECTRODE ASSEMBLY FOR ELECTROOSMOTIC PUMP, ELECTROOSMOTIC PUMP INCLUDING SAME, AND FLUID PUMPING SYSTEM

(71) Applicant: EOFLOW CO., LTD., Seongnam-si, Gyeonggi-do (KR)

(72) Inventors: Young Wook Chang, Seoul (KR); Jae Hong Kim, Seongnam-si Gyeonggi-do (KR); Yongchul Song, Seongnam-si Gyeonggi-do (KR)

(21) Appl. No.: 17/906,256

(22) PCT Filed: Mar. 12, 2021
(86) PCT No.: PCT/KR2021/003115

§ 371 Date: Sep. 13, 2022

(30) Foreign Application Priority Data
Mar. 13, 2020 (KR) 10-2020-0031182

(51) Int. Cl. F04B 19/00

(52) U.S. Cl. CPC F04B 19/006

(57) ABSTRACT

Provided are a membrane-electrode assembly for an electroosmotic pump, the membrane-electrode assembly including a first electrode having a porous structure, containing a first electrochemical reactant, and having applied thereto a (+) or (−) voltage; a first membrane provided on one surface of the first electrode, having a porous structure, and having a (+) or (−) zeta potential; a second electrode provided on one surface of the first membrane opposite to a side of the first electrode, having a porous structure, containing a second electrochemical reactant, and having applied thereto a voltage having a polarity opposite to that of the voltage applied to the first electrode; a second membrane provided on one surface of the second electrode opposite to a side of the first membrane, having a porous structure, and having a zeta potential opposite to that of the zeta potential of the first membrane; and a third electrode provided on one surface of the second membrane opposite to a side of the second electrode, having a porous structure, containing a third electrochemical reactant, and having applied thereto a voltage having a polarity same as that of the voltage applied to the first electrode and a fluid pumping system including the electroosmotic pump.

### TECHNICAL FIELD

The present disclosure relates to a membrane-electrode assembly for an electroosmotic pump and an electroosmotic pump and a system for pumping fluid including the membrane-electrode assembly.

### BACKGROUND ART

An electroosmotic pump is a pump that uses a fluid moving phenomenon that occurs when a voltage is applied to both ends of a capillary tube or a porous membrane.

For the practicability of the electroosmotic pump, it is necessary to generate sufficient pressure and flow rate.

Typically, a flow rate may be achieved by increasing the effective membrane area of an electroosmotic pump. However, in the case of pressure, although increasing an operating voltage is the easiest way to achieve sufficient pressure, power consumption increases and the risk of electrolysis of a working fluid increases.

Also, when an electroosmotic pump is applied to a patch-type drug delivery device or a wearable medical device to be attached to a human body, space availability is limited, and thus it is necessary to provide an electroosmotic pump having a smaller size.

As interest in a method of delivering a drug by implanting a small pump inside the human body increases, researches are being made on small electroosmotic pumps capable of securing sufficient flow rate and pressure.

### DESCRIPTION OF EMBODIMENTS

### Technical Problem

An embodiment of the present disclosure provides a membrane-electrode assembly for an electroosmotic pump.

Another embodiment of the present disclosure provides an electroosmotic pump including the membrane-electrode assembly for an electroosmotic pump.

Another embodiment of the present disclosure provides a fluid pumping system including the electroosmotic pump.

### Technical Solution to Problem

According to an embodiment of the present disclosure, a membrane-electrode assembly for an electroosmotic pump, the membrane-electrode assembly includes a first electrode having a porous structure, containing a first electrochemical reactant, and having applied thereto a (+) or (−) voltage; a first membrane provided on one surface of the first electrode, having a porous structure, and having a (+) or (−) zeta potential; a second electrode provided on one surface of the first membrane opposite to a side of the first electrode, having a porous structure, containing a second electrochemical reactant, and having applied thereto a voltage having a polarity opposite to that of the voltage applied to the first electrode; a second membrane provided on one surface of the second electrode opposite to a side of the first membrane, having a porous structure, and having a zeta potential opposite to that of the zeta potential of the first membrane; and a third electrode provided on one surface of the second membrane opposite to a side of the second electrode, having a porous structure, containing a third electrochemical reactant, and having applied thereto a voltage having a polarity same as that of the voltage applied to the first electrode.

The first electrochemical reactant, the second electrochemical reactant, and the third electrochemical reactant may be the same material or different materials from one another and may each independently include one or more selected from a group consisting of conductive polymers, metals, carbon, and combinations thereof.

The first electrode, the second electrode, and the third electrode may be the same as or different from one another and may each independently have pores of a size from about 0.1 μm to about 500 μm.

The first electrode, the second electrode, and the third electrode may be the same as or different from one another and may each independently have a porosity from 5% μm to 95%.

The first electrode and the third electrode may be electrically connected to each other.

Voltages of different polarities may be alternately supplied to the first electrode, and in correspondence thereto, voltages of different polarities may be alternately supplied to the second electrode and the third electrode.

The first membrane and the second membrane may be the same as or different from each other and may each independently contain one or more selected from a group consisting of silica, alumina, zirconia, magnesium oxide, polymers having (+) potentials, polymers having (−) potentials, and combinations thereof.

The (+) zeta potential may be implemented by using one or more materials selected from a group consisting of alumina, zirconia, magnesium oxide, polymers having (+) potentials, and combinations thereof.

The (+) zeta potential may be implemented by modifying a surface of a membrane with a material having a (+) potential or a functional group having a (+) potential.

The material having a (+) potential or the functional group having a (+) potential may include one or more selected from a group consisting of a primary amine group, a secondary amine group, a tertiary amine group, and combinations thereof.

The (+) zeta potential may be from about 5 mV to about 200 mV.

The (−) zeta potential may be implemented by using one or more materials selected from a group consisting of silica, polymers having (−) potentials, and combinations thereof.

The (−) zeta potential may be implemented by modifying a surface of a membrane with a material having a (−) potential or a functional group having a (−) potential.

The material having a (−) potential or the functional group having a (−) potential may include one or more selected from a group consisting of a carboxyl group, a sulfonic acid group, and combinations thereof.

The (−) zeta potential may be from about −5 mV to about −200 mV.

The first membrane and the second membrane may be the same as or different from one another and may each independently have pores of a size from about 0.05 μm to about 1 μm.

The first membrane and the second membrane may be the same as or different from one another and may each independently have a porosity from 5% to 95%.

The membrane-electrode assembly for an electroosmotic pump may further include an n-th membrane-electrode unit including an (n+2)-th membrane provided on a surface of an (n+2)-th electrode opposite to the side of an (n+1)-th membrane, having a porous structure, and having a zeta potential having the same polarity as that of an n-th membrane; and an (n+3)-th electrode provided on a surface of the (n+2)-th membrane opposite to the side of the (n+2)-th electrode, having a porous structure, containing an (n+3)-th electrochemical reactant, and having applied thereto a voltage of a polarity opposite to that of a voltage applied to an n-th electrode, wherein n is an integer equal to or greater than 1. When n is an integer k equal to or greater than 2, the membrane-electrode assembly for an electroosmotic pump may further include a first membrane-electrode unit and a second membrane-electrode unit to a k-th membrane-electrode unit.

The n may be an integer from 1 to 50.

In the membrane-electrode assembly for an electroosmotic pump, odd-numbered electrodes may be electrically connected to one another. In the membrane-electrode assembly for an electroosmotic pump, even-numbered electrodes may be electrically connected to one another.

Voltages of different polarities may be alternately supplied to the first electrode, and in correspondence thereto, voltages of different polarities may be alternately supplied to the odd-numbered electrodes and the even-numbered electrodes.

Another embodiment of the present disclosure provides an electroosmotic pump including the membrane-electrode assembly for an electroosmotic pump.

Another embodiment of the present disclosure provides a fluid pumping system including the electroosmotic pump.

### Advantageous Effects of Disclosure

A membrane-electrode assembly for an electroosmotic pump according to the present disclosure has a multi-stage structure using a plurality of membranes. However, by sharing electrodes between membranes, the structure of the membrane-electrode assembly may be simplified to reduce the manufacturing cost, the size of the membrane-electrode assembly may be reduced to improve space utilization efficiency, and sufficient pressure or flow rate may be generated.

### BRIEF DESCRIPTION OF DRAWINGS

FIG. 1 is a configuration diagram schematically showing a membrane-electrode assembly for an electroosmotic pump according to an embodiment of the present disclosure.

FIG. 2 is a configuration diagram schematically showing an electroosmotic pump according to another embodiment of the present disclosure.

FIG. 3 is a graph showing respective changes in generated pressures when electroosmotic pumps manufactured according to Embodiment 1 and Comparative Example 1 are driven.

FIG. 4 is a graph showing respective changes in generated pressures when electroosmotic pumps manufactured according to Embodiment 2 and Comparative Example 2 are driven.

### MODE OF DISCLOSURE

Hereinafter, exemplary embodiments of the present disclosure will be described in detail with reference to the accompanying drawings. The present disclosure may, however, be embodied in many different forms and should not be construed as limited to the embodiments set forth herein.

In order to clearly express various layers and regions in the drawings, the thicknesses are enlarged. Throughout the specification, like reference numerals are assigned to like elements. When an element is said to be “on one surface of,” “on,” or “on the surface of” another element, it does not only mean “immediately adjacent to” the other element, but also includes the case where an intervening element is present therebetween.

In addition, unless explicitly described to the contrary, the word “include” will be understood to imply the inclusion of stated elements but not the exclusion of any other elements. Also, “combination” means mixing, alloying, polymerization, or copolymerization.

Throughout the specification, unless otherwise stated, when the terms to electrode(s), membrane(s), membrane-electrode unit(s), and electrochemical reactant(s) each collectively refer to odd-numbered and even-numbered electrodes, odd-numbered and even-numbered membranes, odd-numbered and even-numbered membrane-electrode units, and odd-numbered and even-numbered electrochemical reactants.

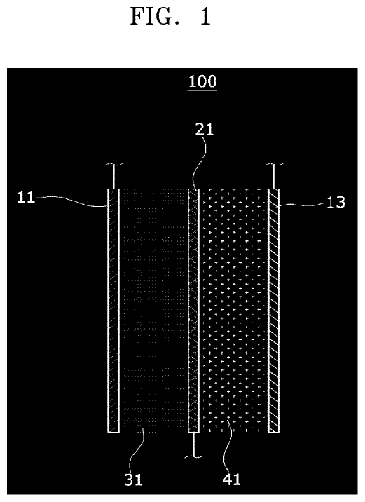

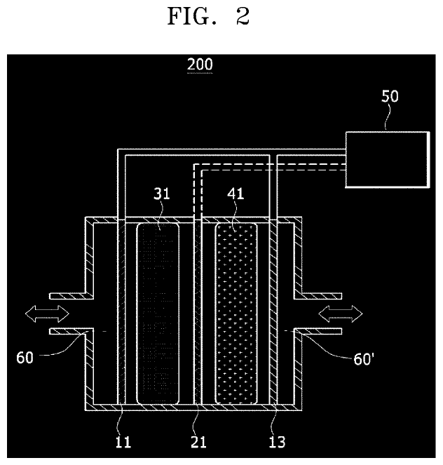

Hereinafter, a membrane-electrode assembly for an electroosmotic pump and an electroosmotic pump according to an embodiment of the present disclosure will be described with reference to FIGS. 1 and 2.

FIG. 1 is a configuration diagram schematically showing a membrane-electrode assembly for an electroosmotic pump according to an embodiment of the present disclosure, and FIG. 2 is a configuration diagram schematically showing an electroosmotic pump according to another embodiment of the present disclosure.

Referring to FIG. 1, a membrane-electrode assembly 100 for an electroosmotic pump has a porous structure, includes a first electrochemical reactant, and includes a first electrode 11 to which a (+) or (−) voltage is applied; a first membrane 31 provided on one surface of the first electrode 11, having a porous structure, and having a (+) or (−) zeta potential; a second electrode 21 provided on one surface of the first membrane 31 opposite to the side of the first electrode 11, having a porous structure, containing a second electrochemical reactant, and having applied thereto a voltage having a polarity opposite to that of the voltage applied to the first electrode 11; a second membrane 41 provided on one surface of the second electrode 21 opposite to the side of the first membrane 31, having a porous structure, and having a zeta potential having a polarity opposite to that of zeta potential of the first membrane 31; and a third electrode 13 provided on one surface of the second membrane 41 opposite to the side of the second electrode 21, having a porous structure, containing a third electrochemical reactant, and having applied thereto a voltage having the same polarity as the voltage applied to the first electrode 11.

Since the polarity of the voltage applied to the first electrode 11 and the polarity of the surface potential of the first membrane 31 are independent from each other, the voltages may have the same polarity or may have different polarities from each other.

The electroosmotic pump membrane-electrode assembly 100 has a multi-stage structure using a plurality of membranes like the first membrane 31 and the second membrane 41, in which the first membrane 31 and the second membrane 41 share the second electrode 21 between the first membrane 31 and the second membrane 41 with each other. As a result, the membrane-electrode assembly 100 for an electroosmotic pump may reduce manufacturing costs by simplifying the structure, may be manufactured in a compact size to improve space utilization efficiency, may generate sufficient pressure or flow rate, may reduce the number of electrodes as compared to a structure in which membranes are connected in series, and may reduce the number of power supply devices to one.

Also, the membrane-electrode assembly 100 for an electroosmotic pump may be configured, such that potentials of the first electrode 11, the first membrane 31, the second electrode 21, the second membrane 41, and the third electrode 13 are arranged according to a certain rule, thereby effectively increasing generated pressure or flow rate while controlling the flow direction of a fluid.

When different voltages, e.g., voltages of different polarities, are supplied to the first electrode 11 and the second electrode 21 and different voltages, e.g., voltages of different polarities, are supplied to the second electrode 21 and the third electrode 13, a voltage difference occurs between the first electrode 11 and the second electrode 21 and a voltage difference occurs between the second electrode 21 and the third electrode 13. Electrochemical reactions occur due to the voltage differences and ions are generated as a result thereof. Generated ions move and drag a fluid together, thereby generating a pressure (pumping force) and a flow rate.

Voltages of different polarities may be alternately applied to the first electrode 11 and the second electrode 21 and to the second electrode 21 and the third electrode 13, wherein alternate application of voltages of different polarities may include application of voltages in directions opposite to each other. Due to the process, the membrane-electrode assembly 100 for an electroosmotic pump generates a pressure (pumping force) through the movement of a fluid, and, at the same time, electrochemical reactants of the first electrode 11, the second electrode 21, and the third electrode 13 may be repeatedly consumed and regenerated.

Hereinafter, the principle that a fluid flow is generated in the membrane-electrode assembly 100 for an electroosmotic pump will be described based on an example.

For example, a fluid flow in the case where, in the membrane-electrode assembly 100 for an electroosmotic pump, the first electrode 11 has a (+) voltage (potential), the first membrane 31 has a (−) voltage (potential), the second electrode 21 has a (−) voltage (potential), the second membrane 41 has a (+) voltage (potential), and the third electrode 13 has a (+) voltage (potential) will be described.

In this case, since the zeta potential of the first membrane 31 is (−), ions having (+) charges (e.g., hydrogen ions (H+)) are collected near the surface of the first membrane 31, and the ions having (+) charges move from the first electrode 11 to the second electrode 21 under the influence of an electric field. At this time, the ions having (+) charges moving under the influence of the electric field attract and drag a surrounding fluid, e.g., water molecules (H₂O), together, and thus the fluid flows in a direction from the first electrode 11 to the second electrode 21.

On the other hand, since the zeta potential of the second membrane 41 between the second electrode 21 and the third electrode 13 is (+), ions having (−) charges (e.g., hydroxide ions (OH−)), are collected near the surface of the second membrane 41, and the ions having (−) charges move in a direction from the second electrode 21 to the third electrode 13 under the influence of an electric field. At this time, the ions having (−) charges moving under the influence of the electric field attract and drag a surrounding fluid, e.g., water molecules (H₂O), together, and thus the fluid flows in the direction from the second electrode 21 to the third electrode 13.

When the two flows of the fluid are combined, a flow of the fluid in a direction from the first electrode 11 to the third electrode 13 appears, and a generated pressure is further increased. The electroosmotic pump may be driven more efficiently by using a pressure and a flow rate generated as described above.

In another example, a fluid flow in the case where, in the membrane-electrode assembly 100 for an electroosmotic pump, the first electrode 11 has a (−) voltage (potential), the first membrane 31 has a (−) voltage (potential), the second electrode 21 has a (+) voltage (potential), the second membrane 41 has a (+) voltage (potential), and the third electrode 13 has a (−) voltage (potential) will be described.

In this case, since the zeta potential of the first membrane 31 is (+), ions having (−) charges (e.g., hydroxide ions (OH−)) are collected near the surface of the second membrane 41, and the ions having (=) charges move from the third electrode 13 to the second electrode 21 under the influence of an electric field. At this time, the ions having (−) charges moving under the influence of the electric field attract and drag a surrounding fluid, e.g., water molecules (H₂O), together, and thus the fluid flows in a direction from the third electrode 13 to the second electrode 21.

On the other hand, since the zeta potential of the first membrane 31 between the second electrode 21 and the first electrode 11 is (−), ions having (+) charges (e.g., hydrogen ions (H+)), are collected near the surface of the first membrane 31, and the ions having (+) charges move in a direction from the second electrode 21 to the first electrode 11 under the influence of an electric field. At this time, the ions having (+) charges moving under the influence of the electric field attract and drag a surrounding fluid, e.g., water molecules (H₂O), together, and thus the fluid flows in the direction from the second electrode 21 to the first electrode 11.

When the two flows of the fluid are combined, a flow of the fluid in a direction from the third electrode 13 to the first electrode 11 appears, and a generated pressure is further increased. The electroosmotic pump may be driven more efficiently by using a pressure and a flow rate generated as described above.

The flows of the fluid described in the above examples are summarized in Table 1 below.

TABLE 1

| | | First Membrane | | Second Membrane | |
|---|---|---|---|---|---|
| | First Electrode | Zeta Potential | Second Electrode | Zeta Potential | Third Electrode |
| | Potential | (Fluid Flow | Potential | (Fluid Flow | Potential |
| | (Voltage) | Direction) | (Voltage) | Direction) | (Voltage) |
| Example | + | −(→) | − | +(→) | + |
| Another | − | −(←) | + | +(←) | − |
| Example | | | | | |

The above examples of describing the principle in which a fluid flows in the membrane-electrode assembly 100 for an electroosmotic pump merely correspond to implementation examples of the present disclosure, and the present disclosure is not limited thereto. The principle in which a fluid flows as described above may be applied as it is to a case where potentials of electrodes and membranes are arranged according to a certain rule in the electroosmotic pump membrane-electrode assembly 100 and may also be applied as it is to a case where voltages are alternately applied to each electrode.

An electrochemical reactant may be a material that may undergo an electrochemical oxidation/reduction reaction.

The first electrochemical reactant, the second electrochemical reactant, and the third electrochemical reactant may be the same material or different materials from one another and may each independently include one or more selected from a group consisting of conductive polymers, metals, carbon, and combinations thereof. However, the first electrochemical reactant, the second electrochemical reactant, and the third electrochemical reactant are not limited thereto and may include other materials as long as the materials may undergo an electrochemical oxidation/reduction reaction.

The conductive polymer may include, but are not limited to, one or more selected from a group consisting of poly(3,4-ethylene dioxythiophene):poly(styrene sulfonate) [PEDOT:PSS], poly(aniline):poly(styrene sulfonate), poly(aniline):camphorsulfonic acid (PANI):CSA), poly(thiophene):poly(styrene sulfonate), polyaniline, polypyrrole, polythiophene, polythionine, quinone-based polymer, and combinations thereof.

The first electrode 11, the second electrode 21, and the third electrode 13 may be the same as or different from one another and may each independently have pores having a size from about 0.1 μm to about 500 μm. In detail, the size of pores may be from about 5 μm to about 300 μm. In more detail, the size of pores may be from about 10 μm to about 200 μm. When the sizes of pores of the first electrode 11, the second electrode 21, and the third electrode 13 are within the above ranges, a fluid and ions may move effectively, and thus the stability, the lifespan characteristics, and the efficiency of the electroosmotic pump may be effectively improved.

The first electrode 11, the second electrode 21, and the third electrode 13 may be the same as or different from one another and may each independently have porosity from about 5% to about 95%. In detail, the porosity may be from about 20% to about 90%. In more detail, the porosity may be from about 50% to about 80%. When the porosity of each of the first electrode 11, the second electrode 21, and the third electrode 13 are within the above ranges, a fluid and ions may move effectively, and thus the stability, the lifespan characteristics, and the efficiency of the electroosmotic pump may be effectively improved.

The first electrode 11 and the third electrode 13 may be electrically connected to each other, for example, through a lead wire. However, as long as the first electrode 11 and the third electrode 13 may be electrically connected, a connection means therebetween is not limited to a lead wire. Therefore, the same voltage may be supplied to the first electrode 11 and the third electrode 13 and effectively move a fluid in a desired direction. Also, a configuration for supplying power may be simplified, and thus the structure of an electroosmotic pump including the same may be simplified and power consumption may be reduced. Also, a generated pressure or a flow rate may be efficiently increased.

Voltages of different polarities may be alternately supplied to the first electrode 11, and in correspondence thereto, voltages of different polarities may be alternately supplied to the second electrode 21 and the third electrode 13 as well. For example, a (+) voltage and a (−) voltage may be alternately supplied to the first electrode 11, and thus a (−) voltage and a (+) voltage may be alternately supplied to the second electrode 21 and a (+) voltage and a (−) voltage may be alternately supplied to the third electrode 13. In this regard, by alternately supplying voltages of different polarities to each electrode, the moving direction of a fluid may be alternately changed between the forward direction and in the reverse direction (or between the reverse direction and forward direction). This principle may be confirmed by referring to the contents described above with respect to Table 1.

The first membrane 31 and the second membrane 41 may be non-conductive.

The first membrane 31 and the second membrane 41 may be the same as or different from each other and may each independently contain one or more selected from a group consisting of silica, alumina, zirconia, magnesium oxide, polymers having (+) potentials, polymers having (−) potentials, and combinations thereof, but is not limited thereto.

The (+) zeta potential may be implemented by using a material exhibiting a (+) potential by itself, and, in detail, may be implemented by using one or more materials selected from a group consisting of alumina, zirconia, magnesium oxide, polymers having (+) potentials, and combinations thereof. However, the present disclosure is not limited thereto.

Meanwhile, the (+) zeta potential may be implemented by modifying the surface of a membrane with a material having a (+) potential or a functional group having a (+) potential, but the present disclosure is not limited thereto. In this case, the inner surface of the membrane may include any one of a material exhibiting a (+) potential, a material exhibiting a (−) potential, a material not exhibiting an electric potential, or a combination thereof.

The material having a (+) potential or the functional group having a (+) potential may include one or more selected from a group consisting of a primary amine group, a secondary amine group, a tertiary amine group, and combinations thereof, but is not limited thereto.

The (+) zeta potential may be from about 5 mV to about 200 mV. in detail, the (+) zeta potential may be from about 10 mV to about 100 mV. In more detail, the (+) zeta potential may be from about 20 mV to about 50 mV. When the (+) zeta potential is within the above-stated ranges, an electroosmotic phenomenon may be effectively caused, thereby driving an electroosmotic pump more efficiently and generating an electric double layer effectively.

The (−) zeta potential may be implemented by using a material exhibiting a (−) potential by itself, and, in detail, may be implemented by using one or more materials selected from a group consisting of silica, polymers having (−) potentials, and combinations thereof, but is not limited thereto.

Meanwhile, the (−) zeta potential may be implemented by modifying the surface of a membrane with a material having a (−) potential or a functional group having a (−) potential, but the present disclosure is not limited thereto. In this case, the inner surface of the membrane may include any one of a material exhibiting a (+) potential, a material exhibiting a (−) potential, a material not exhibiting an electric potential, or a combination thereof.

The material having a (−) potential or the functional group having a (−) potential may include one or more selected from a group consisting of a carboxyl group, a sulfonic acid group, and combinations thereof, but is not limited thereto.

The (−) zeta potential may be from about −5 mV to about −200 mV. in detail, the (+) zeta potential may be from about −10 mV to about −100 mV. In more detail, the (+) zeta potential may be from about −20 mV to about −50 mV. When the (−) zeta potential is within the above-stated ranges, an electroosmotic phenomenon may be effectively caused, thereby driving an electroosmotic pump more efficiently and generating an electric double layer effectively.

The first membrane 31 and the second membrane 41 may be the same as or different from each other and may each independently have pores having a size from about 0.05 μm to about 1 μm. In detail, the size of pores may be from about 0.07 μm to about 0.5 μm. In more detail, the size of pores may be from about 0.1 μm to about 0.15 μm. When the sizes of pores of the first membrane 31 and the second membrane 41 are within the above ranges, a fluid and ions may move effectively, and thus the pressure characteristics, the stability, the lifespan characteristics, and the efficiency of the electroosmotic pump may be effectively improved.

The first membrane 31 and the second membrane 41 may be the same as or different from each other and may each independently have porosity from about 5% to about 95%. In detail, the porosity may be from about 20% to about 80%. In more detail, the porosity may be from about 30% to about 60%. When the porosity of each of the first membrane 31 and the second membrane 41 are within the above ranges, a fluid and ions may move effectively, and thus the pressure characteristics, the stability, the lifespan characteristics, and the efficiency of the electroosmotic pump may be effectively improved.

The membrane-electrode assembly 100 for an electroosmotic pump may further include an n-th membrane-electrode unit including an (n+2)-th membrane provided on a surface of an (n+2)-th electrode opposite to the side of an (n+1)-th membrane, having a porous structure, and having a zeta potential having the same polarity as that of an n-th membrane; and an (n+3)-th electrode provided on a surface of the (n+2)-th membrane opposite to the side of the (n+2)-th electrode, having a porous structure, containing an (n+3)-th electrochemical reactant, and having applied thereto a voltage of a polarity opposite to that of a voltage applied to an n-th electrode, wherein n is an integer equal to or greater than 1. When n is an integer k equal to or greater than 2, the membrane-electrode assembly 100 for an electroosmotic pump may further include a first membrane-electrode unit and a second membrane-electrode unit to a k-th membrane-electrode unit.

As described above, by expanding the structure of a membrane-electrode assembly for an electroosmotic pump, a generated pressure may be increased more effectively.

For example, when n is 1, the membrane-electrode assembly for an electroosmotic pump may include a first electrode having a porous structure, containing a first electrochemical reactant, and having applied thereto a (+) or (−) voltage; a first membrane provided on one surface of the first electrode, having a porous structure, and having a (+) or (−) zeta potential; a second electrode provided on one surface of the first membrane opposite to the side of the first electrode, having a porous structure, containing a second electrochemical reactant, and having applied thereto a voltage having a polarity opposite to that of the voltage applied to the first electrode; a second membrane provided on one surface of the second electrode opposite to the side of the first membrane, having a porous structure, and having a zeta potential of a polarity opposite to that of the zeta potential of the first membrane; a third electrode provided on one surface of the second membrane opposite to the side of the second electrode, having a porous structure, containing a third electrochemical reactant, and having applied thereto a voltage of a polarity same as that of the voltage applied to the first electrode; a third membrane provided on one surface of the third electrode opposite to the side of the second membrane, having a porous structure, and having a zeta potential of a polarity same as that of the zeta potential of the first membrane; and a fourth electrode provided on one surface of the third membrane opposite to the side of the third electrode, having a porous structure, containing a fourth electrochemical reactant, and having applied thereto a voltage of a polarity opposite to that of the voltage applied to the third electrode.

For example, when n is 2, the membrane-electrode assembly for an electroosmotic pump may include a first electrode having a porous structure, containing a first electrochemical reactant, and having applied thereto a (+) or (−) voltage; a first membrane provided on one surface of the first electrode, having a porous structure, and having a (+) or (−) zeta potential; a second electrode provided on one surface of the first membrane opposite to the side of the first electrode, having a porous structure, containing a second electrochemical reactant, and having applied thereto a voltage having a polarity opposite to that of the voltage applied to the first electrode; a second membrane provided on one surface of the second electrode opposite to the side of the first membrane, having a porous structure, and having a zeta potential of a polarity opposite to that of the zeta potential of the first membrane; a third electrode provided on one surface of the second membrane opposite to the side of the second electrode, having a porous structure, containing a third electrochemical reactant, and having applied thereto a voltage of a polarity same as that of the voltage applied to the first electrode; a third membrane provided on one surface of the third electrode opposite to the side of the second membrane, having a porous structure, and having a zeta potential of a polarity same as that of the zeta potential of the first membrane; a fourth electrode provided on one surface of the third membrane opposite to the side of the third electrode, having a porous structure, containing a fourth electrochemical reactant, and having applied thereto a voltage of a polarity opposite to that of the voltage applied to the third electrode; a fourth membrane provided on one surface of the fourth electrode opposite to the side of the third membrane, having a porous structure, and having a zeta potential of a polarity same as that of the zeta potential of the second membrane; and a fifth electrode provided on one surface of the fourth membrane opposite to the side of the fourth electrode, having a porous structure, containing a fifth electrochemical reactant, and having applied thereto a voltage of a polarity opposite to that of the voltage applied to the second electrode.

Even when the value of n is increased, the structure of the membrane-electrode assembly for an electroosmotic pump may be expanded according to the above principle.

The n may be an integer from 1 to 50. In detail, n may be an integer from 2 to 20. In more detail, n may be an integer from 3 to 10. When n is within the above-stated ranges, the pressure of an electroosmotic pump may be controlled as desired.

In the membrane-electrode assembly 100 for an electroosmotic pump, odd-numbered electrodes may be electrically connected to one another, for example, through a lead wire. However, as long as the odd-numbered electrodes may be electrically connected, a connection means therebetween is not limited to a lead wire. Therefore, the same voltage may be supplied to the odd-numbered electrodes and effectively move a fluid in a desired direction. Also, a configuration for supplying power may be simplified, and thus the structure of an electroosmotic pump including the same may be simplified and power consumption may be reduced. Also, a generated pressure or a flow rate may be efficiently increased.

In the membrane-electrode assembly 100 for an electroosmotic pump, even-numbered electrodes may be electrically connected to one another, for example, through a lead wire. However, as long as the even-numbered electrodes may be electrically connected, a connection means therebetween is not limited to a lead wire. Therefore, the same voltage may be supplied to the even-numbered electrodes and effectively move a fluid in a desired direction. Also, a configuration for supplying power may be simplified, and thus the structure of an electroosmotic pump including the same may be simplified and power consumption may be reduced. Also, a generated pressure or a flow rate may be efficiently increased.

Voltages of different polarities may be alternately supplied to the first electrode, and in correspondence thereto, voltages of different polarities may be alternately supplied to the odd-numbered electrodes and the even-numbered electrodes. For example, a (+) voltage and a (−) voltage may be alternately supplied to the first electrode 11, and thus a (−) voltage and a (+) voltage may be alternately supplied to the even-numbered electrodes like the second electrode, the fourth electrode, and a sixth electrode, and a (+) voltage and a (−) voltage may be alternately supplied to the odd-numbered electrodes like the third electrode and the fifth electrode. In this regard, by alternately supplying voltages of different polarities to each electrode, the moving direction of a fluid may be alternately changed between the forward direction and in the reverse direction (or between the reverse direction and forward direction). This principle may be confirmed by referring to the contents described above with respect to Table 1.

Hereinafter, an electroosmotic pump including a membrane-electrode assembly for an electro-osmotic pump according to another embodiment of the present disclosure will be described with reference to FIG. 2.

The electroosmotic pump may be formed in a structure commonly used in the art, and an example thereof will be described below. However, the configuration of the electroosmotic pump is not limited thereto. Descriptions identical to those given above with respect to the membrane-electrode assembly 100 for an electroosmotic pump will be omitted.

A power supply unit 50 may include a DC voltage supply unit (not shown) that supplies a DC voltage to each of the first electrode 11, the second electrode 21, and the third electrode 13. Also, the power supply unit 50 may include a voltage direction changer (not shown) that alternately switches the polarity of the DC voltage supplied to each of the first electrode 11 and the second electrode 21 every certain time, and alternately switches the polarity of the DC voltage supplied to each of the second electrode 21 and the third electrode 13 every certain time. Therefore, it is possible to continuously change the polarity of the voltage applied to each of the first electrode 11 and the second electrode 21 to the opposite polarity every certain time and continuously change the polarity of the voltage applied to each of the second electrode 21 and the third electrode 13 to the opposite polarity every certain time.

Fluid path units 60 and 60′ provide paths for a fluid to move in both directions across membranes and electrodes interposed therebetween.

Here, the fluid path units 60 and 60′ may have a shape of a container filled with a fluid, e.g., a cylinder-like shape, but the shape is not limited thereto.

The fluid may fill not only the fluid path units 60 and 60′, but also membranes and electrodes.

The fluid path units 60 and 60′ may have openings to transmit pressure (pumping force). For example, the openings may be formed in one or both of spaces divided by the membranes and the electrodes and provide pressure (pumping force) due to a movement of a fluid to the outside.

Another embodiment of the present disclosure provides a fluid pumping system including the electroosmotic pump. Since the fluid pumping system may have a structure commonly used in the art, detailed descriptions thereof will be omitted.

Hereinafter, the present disclosure will be described in more detail through embodiments and comparative examples, but the embodiments and the comparative examples below are merely for illustrative purposes and are not intended to limit the present disclosure.

### EMBODIMENTS

### Embodiment 1: Fabrication of a Multistage Electroosmotic Pump

A membrane with a (−) zeta potential was manufactured by molding spherical silica with a diameter of 300 nm by using a rectangular mold of 18 mm×8.5 mm and then sintering the same at a high temperature.

A membrane having a (+) zeta potential was manufactured by forming a primary amine group on the surface of silica using a surface modification method.

As an electrode, a porous electrode was prepared by electropolymerizing poly(aniline):poly(styrene sulfonate) on a porous carbon paper and the porous electrode was according to a size.

Membranes and electrodes manufactured as described above were arranged as shown in FIG. 2, and a sustain strip was superimposed on each electrode to apply a voltage from the outside. After a plastic housing with water inlets on both sides was installed to measure the pressure of the electroosmotic pump, the outer portion of the plastic housing was sealed by using an epoxy resin.

### Embodiment 2: Fabrication of a Multistage Electroosmotic Pump

A membrane with a (−) zeta potential was manufactured by molding spherical silica with a diameter of 300 nm by using a rectangular mold of 18 mm×8.5 mm and then sintering the same at a high temperature.

A membrane having a (+) zeta potential was manufactured by forming a primary amine group on the surface of silica using a surface modification method.

As an electrode, poly(aniline):poly(styrene sulfonate) was electropolymerized on a porous carbon paper and was washed and dried. Next, a solution in which poly(3,4-ethylene dioxythiophene):poly(styrene sulfonate) particles were dispersed was applied thereon through dip coating and dried in an oven, and a dried electrode was cut according to a size.

Membranes and electrodes manufactured as described above were arranged as shown in FIG. 2, and a sustain strip was superimposed on each electrode to apply a voltage from the outside. After a plastic housing with water inlets on both sides was installed to measure the pressure of the electroosmotic pump, the outer portion of the plastic housing was sealed by using an epoxy resin.

### Comparative Example 1: Fabrication of a Single Electroosmotic Pump

The electrodes manufactured in Embodiment 1 were used. The electrodes were placed on both ends of a silica membrane having a (−) zeta potential, a suspension strip was superimposed on each electrode to apply a voltage from the outside, and, to measure the pressure of the electroosmotic pump, a plastic housing with water inlets on both sides was installed, and the outer portion of the plastic housing was sealed by using an epoxy resin.

### Comparative Example 2: Fabrication of a Single Electroosmotic Pump

Except that the electrodes prepared in Embodiment 2 were used, the electroosmotic pump was manufactured in the same manner as in Comparative Example 1.

### Test Example 1: Performance Evaluation of Electroosmotic Pump (Generated Pressure)

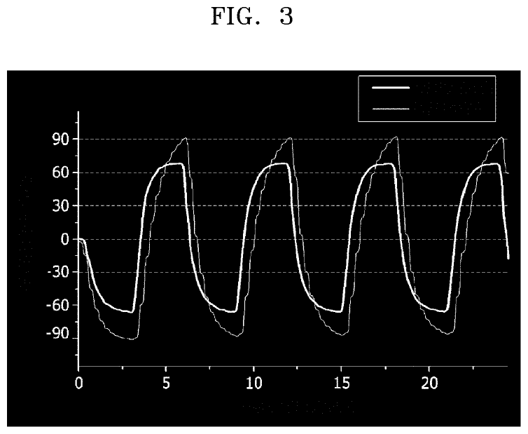

The pressure of a fluid generated when a potential difference of 2.5V was alternately applied for 25 seconds to each electrode of each electroosmotic pump manufactured in Embodiment 1 and Comparative Example 1 with a cycle of 3 seconds was evaluated, and results thereof are shown in FIG. 3.

As shown in FIG. 3, it may be seen that the pressure generated by the electroosmotic pump manufactured in Comparative Example 1 is at the maximum of 68 kPa, while the pressure generated by the electroosmotic pump manufactured in Embodiment 1 is at the maximum of 92 kPa. In other words, it may be seen that the pressure generated by the electroosmotic pump manufactured in Embodiment 1 is improved by about 35% or more as compared to the pressure generated by the electroosmotic pump manufactured in Comparative Example 1.

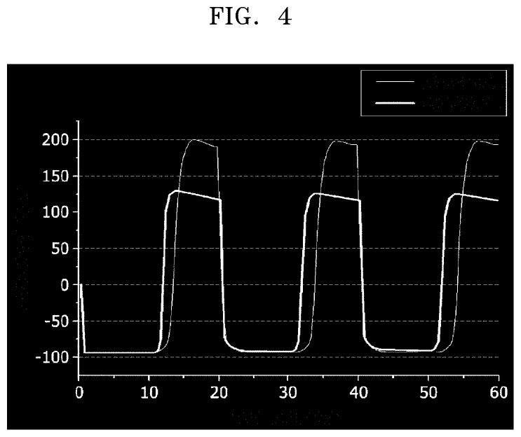

The pressure of a fluid generated when a potential difference of 4V was alternately applied for 60 seconds to each electrode of each electroosmotic pump manufactured in Embodiment 2 and Comparative Example 2 with a cycle of 10 seconds was evaluated, and results thereof are shown in FIG. 4.

As shown in FIG. 4, it may be seen that the pressure generated by the electroosmotic pump manufactured in Comparative Example 2 is at the maximum of 130 kPa, while the pressure generated by the electroosmotic pump manufactured in Embodiment 2 is at the maximum of 200 kPa. In other words, it may be seen that the pressure generated by the electroosmotic pump manufactured in Embodiment 2 is improved by about 55% or more as compared to the pressure generated by the electroosmotic pump manufactured in Comparative Example 2.

### INDUSTRIAL APPLICABILITY

Although example embodiments of the present disclosure have been described above, it is obvious that the present disclosure is not limited thereto, and various modifications and variations may be made within the scope of the claims, the detailed description of the disclosure, and the accompanying drawings, and the modifications and the variations also fall within the scope of the present disclosure.

1. A membrane-electrode assembly for an electroosmotic pump, the membrane-electrode assembly comprising:
a first electrode having a porous structure and containing a first electrochemical reactant, and configured for applying thereto a positive (+) or negative (−) voltage;
a first membrane provided on one surface of the first electrode, having a porous structure, and having a positive (+) or negative (−) zeta potential;
a second electrode provided on one surface of the first membrane opposite to a side of the first electrode, having a porous structure, containing a second electrochemical reactant, configured for applying thereto a voltage having a polarity opposite to that of the voltage applied to the first electrode;
a second membrane provided on one surface of the second electrode opposite to a side of the first membrane, having a porous structure, and having a zeta potential opposite to that of the zeta potential of the first membrane; and
a third electrode provided on one surface of the second membrane opposite to a side of the second electrode, having a porous structure, containing a third electrochemical reactant, and configured for applying thereto a voltage having a polarity same as that of the voltage applied to the first electrode.

2. The membrane-electrode assembly of claim 1, wherein the first electrochemical reactant, the second electrochemical reactant, and the third electrochemical reactant are the same material or different materials from one another and each independently include one or more selected from the group consisting of conductive polymers, metals, carbon, and combinations thereof.

3. The membrane-electrode assembly of claim 1, wherein the first electrode, the second electrode, and the third electrode are the same as or different from one another and each independently have pores of a size from about 0.1 μm to about 500 μm.

4. The membrane-electrode assembly of claim 1, wherein the first electrode, the second electrode, and the third electrode are the same as or different from one another and each independently have a porosity from 5% μm to 95%.

5. The membrane-electrode assembly of claim 1, wherein the first electrode and the third electrode are electrically connected to each other.

6. The membrane-electrode assembly of claim 1, wherein the voltages of different polarities are alternately supplied to the first electrode, and in correspondence thereto, the voltages of different polarities are alternately supplied to the second electrode and the third electrode.

7. The membrane-electrode assembly of claim 1, wherein the first membrane and the second membrane are the same as or different from each other and each independently contain one or more selected from the group consisting of silica, alumina, zirconia, magnesium oxide, polymers having (+) potentials, polymers having (−) potentials, and combinations thereof.

8. The membrane-electrode assembly of claim 1, wherein the (+) zeta potential is implemented by using one or more materials selected from the group consisting of alumina, zirconia, magnesium oxide, polymers having (+) potentials, and combinations thereof.

9. The membrane-electrode assembly of claim 1, wherein the (+) zeta potential is implemented by modifying a surface of a membrane with a material having a (+) potential or a functional group having a (+) potential.

10. The membrane-electrode assembly of claim 9, wherein the material having the (+) potential or the functional group having the (+) potential comprises one or more selected from the group consisting of a primary amine group, a secondary amine group, a tertiary amine group, and combinations thereof.

11. The membrane-electrode assembly of claim 1, wherein the (+) zeta potential is from about 5 mV to about 200 mV.

12. The membrane-electrode assembly of claim 1, wherein the (−) zeta potential is implemented by using one or more materials selected from the group consisting of silica, polymers having (−) potentials, and combinations thereof.

13. The membrane-electrode assembly of claim 1, wherein the (−) zeta potential is implemented by modifying a surface of a membrane with a material having a (−) potential or a functional group having a (−) potential.

14. The membrane-electrode assembly of claim 13, wherein the material having the (−) potential or the functional group having the (−) potential comprises one or more selected from the group consisting of a carboxyl group, a sulfonic acid group, and combinations thereof.

15. The membrane-electrode assembly of claim 1, wherein the (−) zeta potential is from about −5 mV to about −200 mV.

16. The membrane-electrode assembly of claim 1, wherein the first membrane and the second membrane are the same as or different from one another and each independently have pores of a size from about 0.05 μm to about 1 μm.

17. The membrane-electrode assembly of claim 1, wherein the first membrane and the second membrane are the same as or different from one another and each independently have a porosity from 5% to 95%.

18. The membrane-electrode assembly of claim 1, further comprising an n-th membrane-electrode unit comprising:
an (n+2)-th membrane provided on one surface of an (n+2)-th electrode opposite to a side of an (n+1)-th membrane, having a porous structure, and having a zeta potential same as that of a zeta potential of an n-th membrane; and
an (n+3)-th electrode provided on one surface of the (n+2)-th membrane opposite to a side of the (n+2)-th electrode, having a porous structure, containing an (n+3)-th electrochemical reactant, and having applied thereto a voltage having a polarity opposite to that of a voltage applied to the n-th electrode,
wherein n is an integer equal to or greater than 1, and, when n is an integer k equal to or greater than 2, the membrane-electrode assembly further comprises a first membrane-electrode unit and a second membrane-electrode unit to a k-th membrane-electrode unit.

19. The membrane-electrode assembly of claim 18, wherein odd-numbered electrodes are electrically connected to one another, and even-numbered electrodes are electrically connected to one another.

20. The membrane-electrode assembly of claim 18, wherein voltages of different polarities are alternately supplied to the first electrode, and in correspondence thereto, voltages of different polarities are alternately supplied to the odd-numbered electrodes and the even-numbered electrodes.
